(19) United States
(12) Patent Application Publication
(10) Pub. No.: US 2004/0191317 A1
(43) Pub. Date: Sep. 30, 2004

(54) STYPTIC PENCIL OR STICK HOUSED IN A TUBULAR DISPENSER

(76) Inventor: Jeffrey Kaplan, Fort Lauderdale, FL (US)

Correspondence Address: MALIN HALEY AND DIMAGGIO, PA, 1936 S ANDREWS AVENUE, FORT LAUDERDALE, FL, 33316, US

(21) Appl. No.: 10/709,242

(22) Filed: Apr. 23, 2004

Related U.S. Application Data
(63) Continuation-in-part of application No. 10/350,943, filed on Jan. 27, 2003.

(51) Int. Cl. A61K 9/14; A61L 15/16

(52) U.S. Cl. 424/486

(57) ABSTRACT

A moisture-proof sealable styptic pencil dispenser including a styptic pencil, a sealable cover and holder with a base having a top surface adapted for holding and enclosing the styptic pencil. The holder also has an inner retainer having a top end located generally circumferentially around the base and a manual actuator for adjusting the distance of the base to the top end of the retainer located at the bottom end of the dispenser. The adjusting actuator has a grip, whereby the distance of the base to the inner retainer is adjusted by the rotation or other manipulation of the grip to allow only the styptic pencil tip to be exposed to prevent moisture from reaching the pencil body.

### BACKGROUND OF INVENTION

1. Field of the Invention

This invention relates to a styptic pencil or stick dispenser comprising a styptic composition in stick or pencil form and a tubular, closeable sealable housing that retains the styptic material and through which the styptic material is manually advanced for use to curtail bleeding from minor nicks and cuts. More particularly, this invention relates to a dispenser for a styptic pencil that is moisture sealed and waterproof to prevent deterioration of the styptic pencil.

2. Description of Related Art

Nicks and cuts on the chin, face and other areas are a virtually inevitable consequence of regular blade shaving. Nicks and cuts can also be a significant problem for women when shaving their legs and underarms. Shaving cuts, especially in the area of the face tend to bleed profusely, and it can be quite difficult to halt the flow of blood.

The use of styptic or astringent compositions is well established in the art. The principal astringent chemicals are compounds of aluminum, potassium, zinc, manganese, iron, bismuth and other chemical groups that contain these metals (such as permanganates). Although some astringent substances are potent chemicals, they do not enter human body cells, which is their virtue. Rather, all their activity occurs at cell surface where they block noxious substances from entering the cell or the space between the cells. They coagulate body chemicals so that they form clots, crusts or other solid deposits.

To go back to the original example of the man who cuts himself while shaving, an astringent styptic cast pencil is normally used. A small area near the top of the styptic pencil is manually placed against the skin area where the cut occurs. The end tip may be slightly wetted to dissolve styptic material onto the skin cut. These styptic pencils have been used since 1890 and usually consist of 80-90% aluminum or potassium sulfate and 10-20% of an inert filler. Currently, styptic compositions available to the public are the generic molded cast solid styptic pencil, a stick design or a powdered styptic composition.

Typical styptic compositions in pencil and stick forms are set forth in U.S. Pat. No. 5,718,865 to Askew, U.S. Pat. No. 819,901 to Maschal, U.S. Pat. No. 459,738 to Black and U.S. Pat. No. 497,659 to Harned. These cited examples all show various improved ways of dealing with the manufacture and production procedures of making styptic pencils or sticks but do not deal with the problems associated with storing and preserving said styptic pencils or sticks when used by the general public.

In addition, U.S. Pat. No. 5,955,122 discloses a styptic pencil with aluminum and other ingredients. However, a dispenser has not been contemplated for best use with the type of formulation for a pencil as described therein.

Currently, the generic solid molded styptic pencil or stick designs are manufactured using conventional techniques such as mechanical devices, nozzle filling or flood filling and then simply placed in a flimsy covered plastic vial. Using mechanical device, solid pieces of alum are cut and ground into pencil or stick shapes. In the nozzle fill method, a homogeneous styptic composition is dispensed from a filling nozzle directly into molds. In the flood fill method, the molds are placed in an opening of a tray and the styptic composition is poured into the tray thereby flooding the tray and in turn filling the molds.

When needed, the user in the past removed the entire exposed pencil or stick from the plastic vial and applied water to wet the tip edge, which in turn softens the styptic composition. Once the tip is wet, the user then applies the styptic composition to the wound. Since the user is normally shaving and is in the bathroom, the entire exposed styptic pencil or stick gets wet from the user's hands and the ambient humidity. After use, the entire exposed styptic pencil or stick is supposed to be dried and placed back into the plastic vial for future use. Unfortunately, when the entire exposed styptic pencil or stick is repeatedly wetted and stored, the entire exposed pencil or stick begins to crumble, deteriorate and normally adheres to the inside of the plastic vial container thus causing further damage when removal is attempted. Even if the user dries the styptic pencil or stick properly, within a few weeks of normal use the entire styptic pencil or stick becomes so deteriorated it must be thrown away.

In various attempts to obviate these problems, several different types of styptic devices have been introduced. These consist of styptic bandages in U.S. Pat. No. 3,113,568 to Robins, U.S. Pat. No. 4,022,203 to Ackley and U.S. Pat. No. 4,233,976 to Dunshee. In addition, styptic creams have been introduced in U.S. Pat. Nos. 4,166,108 and 5,279,837.

However, none of these unique styptic devices have been accepted by the mass public. Thus, a need exists for a styptic pencil or stick dispenser comprising a styptic pencil or stick mounted in a tubular, sealable housing that permits the styptic pencil or stick to be advanced or ejected for use without exposing the entire stick to moisture or wetness.

### SUMMARY OF INVENTION

In the present invention, the foregoing difficulties are obviated in that there is provided a styptic composition housed in a moisture-proof, sealable tubular dispenser thereby keeping water and moisture from the styptic composition until ready to use by advancing or ejecting said styptic composition end tip from the tubular dispenser.

It is an object of the invention to provide a novel styptic pencil or stick dispenser which allows for the advantages of a pencil protected from wetness and high humidity.

It is another object of the invention to provide a novel styptic pencil or stick dispenser comprising a styptic pencil or stick composition and a moisture-proof sealable tube or sleeve which houses the styptic pencil or stick composition and through which the styptic pencil or stick composition is advanced.

It is still another object of the invention to provide a novel styptic pencil or stick dispenser which will keep the enclosed styptic composition stable and free from water and moisture.

It is yet another object of the invention to provide a novel styptic pencil or stick dispenser which is simple to make, inexpensive and easy to use.

It is still another object of this invention to provide an inexpensive solution to reduce or eliminate the unwanted brittle, drying, deteriorating and sticking properties associated with current exposed styptic pencils or sticks available to the public.

In accordance with these and other objects which will become apparent hereinafter, the instant invention will now be described with particular reference to the accompanying drawings.

### BRIEF DESCRIPTION OF DRAWINGS

FIG. 1 shows a perspective view of the preferred embodiment of the invention in use on a person's leg.

FIG. 2A is a side elevational view of the preferred embodiment of the invention.

FIG. 2B is a side elevational view of the embodiment of the invention shown in FIG. 2A with the cap removed.

FIG. 2C is an exploded cross sectional side elevational view of the embodiment of the invention shown in FIG. 2A.

FIG. 2D is a cross sectional elevational view of the cap of the embodiment of the invention shown in FIG. 2A.

FIG. 3 is a cross-sectional view of the preferred embodiment of the invention taken along line 3-3 of FIG. 4.

FIG. 4 is a partial cross-sectional view of the preferred embodiment of the invention taken along line 4-4 of FIG. 3.

FIG. 5 is a cut-away cross sectional view of the base and partial pencil of the invention.

FIG. 6 is an exploded side elevational view of an alternative embodiment of the invention.

### DETAILED DESCRIPTION

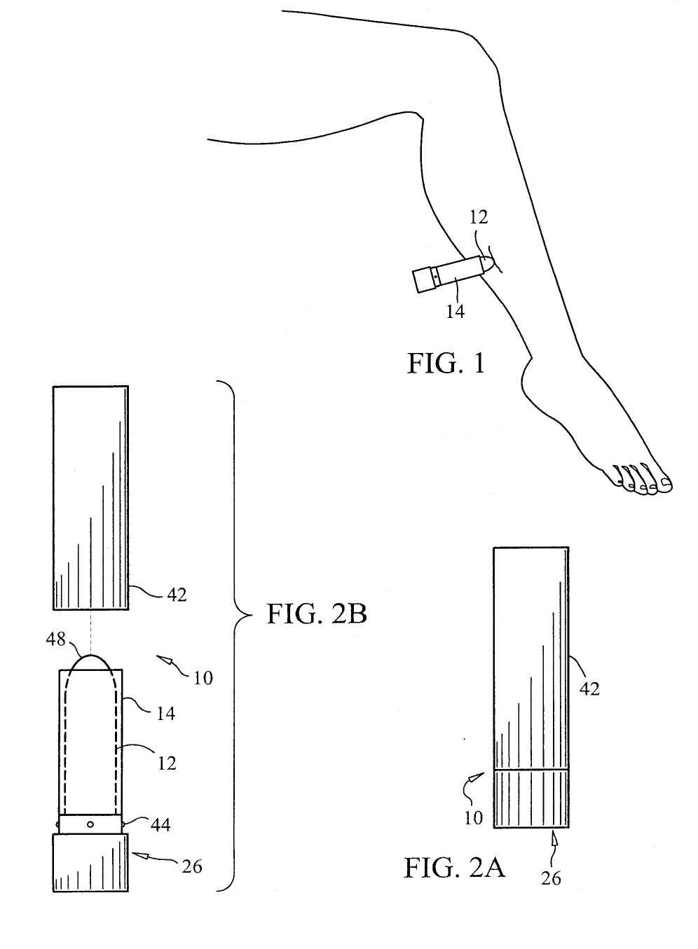

Referring now to the drawings, FIG. 1 shows the preferred embodiment of the instant invention, which comprises a styptic pencil dispenser 10, comprising a styptic pencil 12, shown with the cap removed for use.

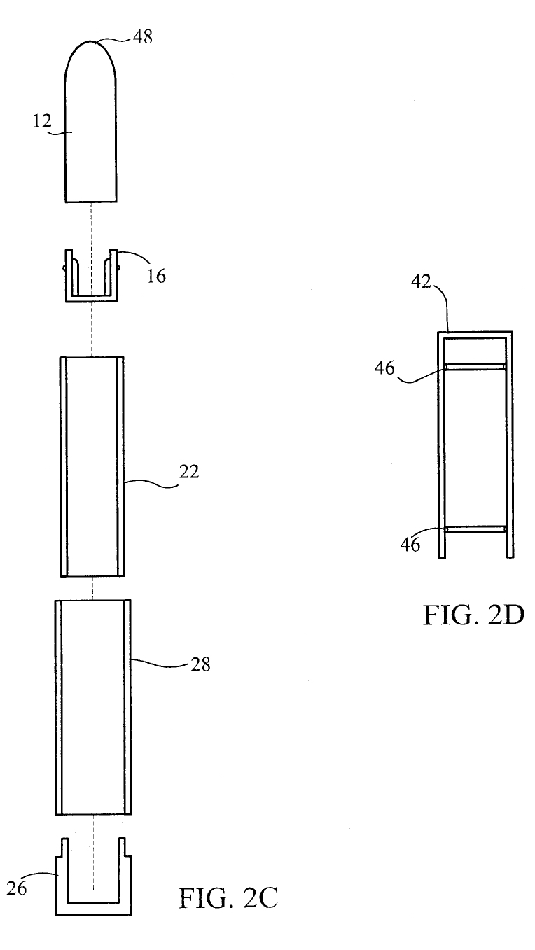

FIGS. 2A-2D shows the invention 10 including a holder 14 and a sealable cap or cover 42. In the closed condition, the dispenser 10 protects the contents inside. Thus, the styptic pencil 12 is protected from moisture, water and high humidity because cap 42 is sealably connected to the holder 12 in such a way as to prevent any moisture from permeating the dispenser. Only the tip 48 of the styptic pencil 12 is exposed to the environment. The styptic material of the pencil 12 preferably includes a water soluble resin such as polyethylene glycol to obtain the preferred consistency and glycerine which prevents the composition from sticking to the housing.

FIGS. 2A and 2B show the location of the styptic pencil 12 with only a small tip end exposed during use with the sealable cap 42 removed. The cap 42 inside diameter is sized to fit very tightly on the outside diameter of the holder 12 to act as a moisture seal. One or more O-ring seals or annular rings 46 on the inside of the cap as shown in FIG. 2D can also be used to form a moisture proof seal with the cover. In addition, the tolerance of the fit of the cap 42 onto the holder 12 preferably enhances the protection of the styptic pencil against moisture. It also may be preferred that the cover is clear so the pencil may be viewed through the cap or cover.

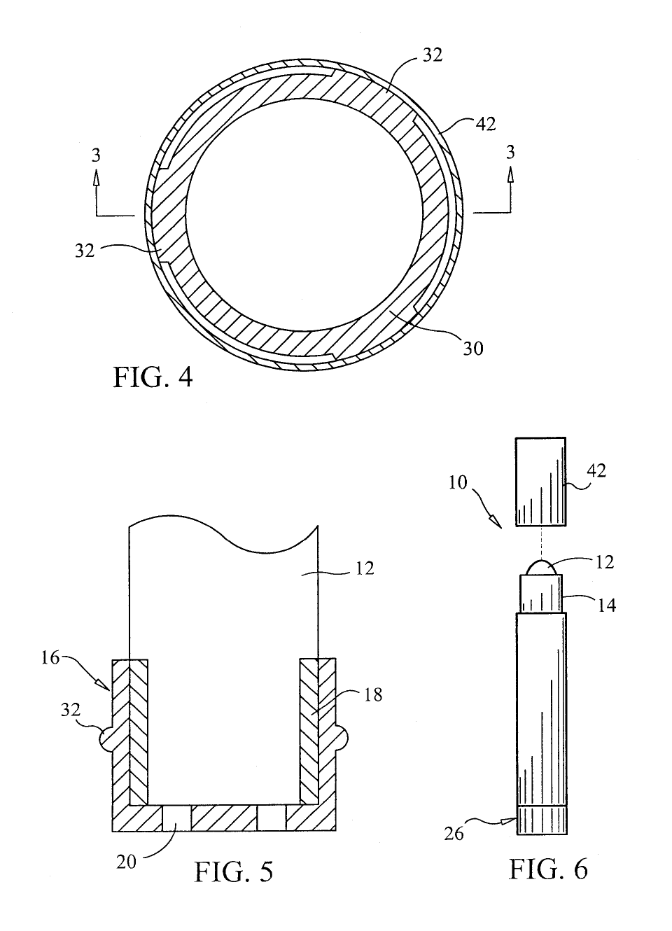

As shown in FIG. 2C and FIGS. 3-6, the holder 14 includes an annular tubular base 16 having a top surface 18 adapted for holding the styptic pencil 12.

The base 16 is generally adapted for holding a styptic pencil as described herein. As shown in FIG. 6, preferably includes elements for retaining the styptic pencil on the base, such as one or more protruding ribs 18 for engagement with the styptic pencil 12, and at least one aperture 20. The friction between the pencil 12 and the retention ribs keep the pencil in place on the base.

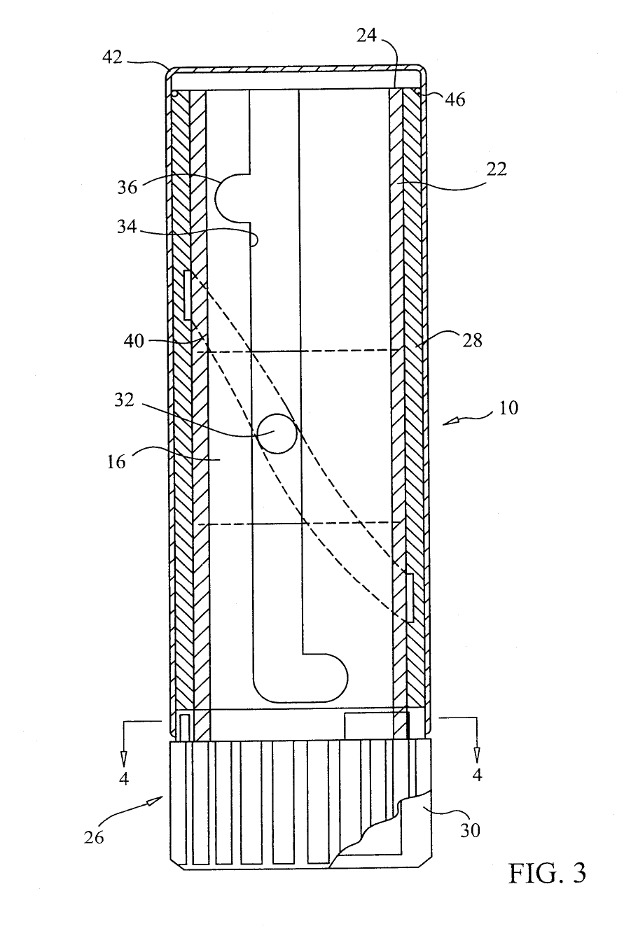

As shown in FIGS. 2C and 3, the invention 10 includes an inner retainer 22 having a top end 24 located generally circumferentially around the base 16. A manual actuator for adjusting the distance of the base to the top end of the retainer, shown generally at 26, is located at the bottom end of the dispenser 10.

The actuator for adjusting 26 includes an outer generally cylindrical shell 28 located peripherally around the inner retainer 22, whereby the inner retainer 22 is movable within the outer shell 28. It is preferred that the motion by the inner retainer 22 is generally rotational. In addition, the actuator for adjusting further includes a grip 30, which is preferred to be rotatable. Alternatively, the grip may be stationary with a movable attachment for adjusting the height of the styptic pencil. The distance of the base 16 to the top end of the inner retainer 22 is adjusted by the rotation or other movement of the grip 30. However, the mechanical movement for adjustment of the tip height of the styptic pencil may alternatively be linear.

In the preferred embodiment of the invention, the styptic pencil activator of the invention 10 activates to advance or retract the styptic pencil through the interaction between the base 16, the inner retainer 22 and the outer shell 28. The base 16 includes at least one protrusion 32. The inner retainer 22 includes a vertical slot 34. The slot preferably includes a bottom end point 36 and a top end point 38 to provide limits for the distance which the base 16 may travel. The outer shell 28 includes at least one helical depression 40 at least partway through the outer shell 28. Thus, the protrusion passes through the slot 34 and engages the depression 40. Frictional engagement between the outer shell 28 and the inner retainer 22 allow the user to control the distance traveled by the base 16. For best results, the grip 30 is engaged with or an intrinsic part of the inner retainer 22. However, other means for controlling the means of adjustment for the base are contemplated.

The dispenser includes a sealable cap 42 for protecting the pencil 12 from exposure to unwanted moisture or wetness which can deteriorate the pencil. As shown in FIGS. 2 and 7, the cap 42 may surround all of or a portion of the outer shell 28. It is also preferred that the cover is frictionally engaged with the activator so that the distance of the base 14 from the top 24 of the inner retainer 22 is generally fixed. It is also preferred that the activator includes protrusions 44 for engagement with the cap 42, as shown in FIG. 2B. However, the cover may alternatively have depressions for frictional engagement with the grip 30.

The holder 14 may be used as described above to protect the pencil 12 from unwanted wetting, whether from accident in the bathroom or from the ambient humidity. Furthermore, the extendable sleeve prevents unwanted contact between the pencil 12 and the cap 42, which would otherwise facilitate the deterioration of the pencil 12. In addition, the holder 14, with its sides surrounding the pencil 12, helps reduce exposure of the pencil to unwanted moisture with or without the cap 42.

The term “styptic pencil or stick composition” is defined as a a pencil or composition including a solid or semi-solid malleable styptic composition that is sufficiently solid so as to substantially retain its shape when advanced or ejected from an open end of the container, while capable of leaving a desired residue when applied be means such as wetting.

The styptic pencil or stick composition is generally well established in the art comprising ingredients such as aluminum sulfate, potassium sulfate, zinc, manganese, iron, titanium dioxide, bismuth and other elements or chemical groups that contain these metals (such as permanganates). Well known manufacturing processes such as nozzle filling or flood filling may be utilized. In addition, by adding plasticizers and other additives, fragrances, skin conditioners moisturizers and the like can be included in the manufacturing process.

The invention further comprises a styptic pencil or stick composition storage and dispensing unit comprising:

a tubular housing, said housing providing an interior space for a styptic pencil or stick composition, said housing providing an interior base defining a bottom ejector, said bottom ejector communicating with said interior styptic pencil or stick composition;

a sealable cover or cap to keep out moisture or wetness, said cover or cap attached or removable to said housing, covering and exposing the styptic pencil or stick composition; and

ejector for controllably advancing or ejecting the styptic pencil tip through said housing such as by mechanical rotation or mechanical force without rotation.

In addition, it may be preferred that the styptic pencil or stick composition is substantially wholly contained inside said housing and is filled by conventional manufacturing processes such as mechanical device, nozzle filling, flood filling or other known manufacturing methods.

Furthermore, the housing unit is designed and inexpensively manufactured to keep said styptic pencil or stick composition dry, free from water and moisture.

The styptic pencil or stick composition contained in the housing is manufactured using astringent chemicals or natural ingredients which coagulate blood, such as alum, aluminum sulfate, aluminum acetate, potassium sulfate, potassium alum, ammonium alum, potassium nitrate, ammonium chloride hexahydrate, titanium dioxide, zinc sulfate, ferric sulfate, manganese, iron alum, caesium alum, chrome alum, chromoselenic alum, bismuth, copper sulfate, iron sulfate, tannin acid and other chemical or natural organic groups that contain these metals such as permanganates. In addition, the styptic pencil or stick composition can be a solid or semi-solid malleable styptic composition which retains its shape when advanced or ejected from an open end of the tubular housing, while capable of leaving a desired residue when applied by wetting. Also, the styptic pencil or styptic composition may preferably contain additional ingredients such as fragrances, skin conditioning agents, moisturizers, preservatives, emollients, surfactants, medicaments, colorants, flavorants, perfumes, polyol plasticizers, glycerol, sorbitol, borax, antiseptic compounds, hamamelis water NF X1 (witch hazel), aloe extracts, vitamins, plant/herb derivatives and water.

The instant invention has been shown and described herein in what is considered to be the most practical and preferred embodiment. It is recognized, however, that departures may be made therefrom within the scope of the invention and that obvious modifications will occur to a person skilled in the art.

What is claimed is:

1. A styptic pencil dispenser, comprising:
a styptic pencil having a predetermined diameter, and
a styptic pencil holder, including:
an annular tubular base having cylindrical cavity sized in diameter approximately equal to said styptic pencil diameter to retain said styptic pencil; and
an inner retainer sleeve having a top end located generally circumferentially around the base;
a means for adjusting the distance of the base retaining said styptic pencil to the top end of the retainer located at the bottom end of the dispenser allowing the tip only to be exposed for use; and
a sealable cap sized to fit snug with and over said retaining sleeve to prevent the styptic pencil from moisture and becoming wet during application.

2. The dispenser of claim 1, wherein the base has at least one rib for holding the styptic pencil.

3. The dispenser of claim 1, wherein the means for adjusting comprises an outer generally cylindrical shell located peripherally around the inner retainer, whereby the inner retainer is rotatable within the outer shell.

4. The dispenser of claim 3, wherein the means for adjusting further comprises a rotatable grip, whereby the distance of the base to the inner retainer is adjusted by the rotation of the grip.

5. The dispenser of claim 4, wherein:
the base further includes at least one protrusion,
the inner retainer includes a vertical slot having a bottom endpoint and a top endpoint, and
the outer cylinder includes at least one helical depression at least partway through the outer shell,
whereby the protrusion engages both the slot and the depression and whereby the base is elevated and lowered by movement of the means for adjusting.

6. The dispenser of claim 5, wherein the engagement of the protrusion with the slot and the depression limit the adjustment distance of the base.

7. The dispenser of claim 1, wherein the styptic pencil comprises a water soluble resin.

8. The dispenser of claim 7, wherein the styptic pencil further comprises polyethylene glycol and glycerine.

9. The dispenser of claim 1, wherein the base includes means for retaining the styptic pencil.

10. The dispenser of claim 9, wherein the means for retaining includes at least one rib for engagement with the styptic pencil, and
at least one depression at least partway through the top surface.

11. The dispenser of claim 1, wherein the cap further comprises a means for sealing moisture or wetness from the styptic pencil.

12. The dispenser of claim 1, wherein the means for sealing comprises at least one annular seal.

13. The dispenser of claim 11, wherein the cap is frictionally engaged with the means for adjusting, whereby the styptic pencil is protected from moisture.

14. The dispenser of claim 1, wherein the tolerance of the fit of the cap enhances the protection of the styptic pencil against moisture.

15. The dispenser of claim 1, further comprising protrusions for engagement with the cap whereby exposure of the styptic pencil to moisture is decreased when the cap is in place.

16. A styptic pencil dispenser, comprising:
a styptic pencil, and
a holder, including:
a base having a top surface adapted for holding a styptic pencil; and
an inner retainer having a top end located generally circumferentially around the base; and
means for adjusting the distance of the base retaining said styptic pencil to the top end of the retainer located at the bottom end of the dispenser; and
a cover for protecting the styptic pencil from moisture or wetness.

17. The dispenser of claim 16, wherein the cover frictionally engages with the means for adjusting.

18. A styptic pencil or stick composition storage and dispensing unit comprising:
tubular housing sized in length to cover a styptic pencil, said housing providing an interior space for a styptic pencil or stick composition, said housing providing an interior base defining a bottom ejector, said bottom ejector communicating with said interior styptic pencil or stick composition;
a cover, said cover removeably attached to said housing, covering and exposing the styptic pencil or stick composition; and
an ejector for controllably advancing or ejecting the styptic pencil or stick composition through said housing such as by mechanical rotation or mechanical force without rotation.

19. The styptic pencil or stick composition storage and dispensing unit as set forth in claim 16, wherein the styptic pencil or stick composition is substantially wholly contained inside said housing and is filled by conventional manufacturing processes.

20. The styptic pencil or stick composition storage and dispensing unit of claim 16, wherein the housing unit resembles tubular lip balms, sun-screens, deodorants or glue stick designs.
